(19) United States
(12) Patent Application Publication
(10) Pub. No.: US 2018/0171135 A1
(43) Pub. Date: Jun. 21, 2018

(54) INTERLAYER INSULATING RESIN FILM, INTERLAYER INSULATING RESIN FILM HAVING ADHESIVE AUXILIARY LAYER, AND PRINTED CIRCUIT BOARD

(71) Applicant: INTEL CORPORATION, Santa Clara, CA (US)

(72) Inventors: Aya KASAHARA, Tokyo (JP); Yasuyuki MIZUNO, Tokyo (JP); Hikari MURAI, Tokyo (JP)

(21) Appl. No.: 15/549,655

(22) PCT Filed: Feb. 10, 2016
(86) PCT No.: PCT/JP2016/054032

§ 371 Date: Dec. 18, 2017

(30) Foreign Application Priority Data
Feb. 10, 2015 (JP) 2015-024451

(51) Int. Cl. C08L 63/00; C08L 79/00; C08J 5/18; C07C 279/28; C09J 163/00; C09J 179/00; C08K 3/36; H05K 1/03

(52) U.S. Cl. CPC C08L 63/00; C08L 79/00; C08J 5/18; H05K 1/0373; C09J 163/00; C09J 179/00; C08K 3/36; C07C 279/28

(57) ABSTRACT

Provided are an interlayer insulating resin film, an interlayer insulating resin film having an adhesive auxiliary layer, and a printed circuit board obtained using the interlayer insulating resin film or the interlayer insulating resin film having an adhesive auxiliary layer, with which it is possible to obtain an interlayer insulating layer having exceptional adhesion to a circuit board even after accelerated environmental testing, and exceptional heat resistance, dielectric characteristics, and low thermal expansion. Specifically: an interlayer insulating resin film containing an epoxy resin (A), a cyanate resin (B), and a dicyandiamide (C); an interlayer insulating resin film having an adhesive auxiliary layer, the adhesive auxiliary layer being provided on one surface of the above-mentioned interlayer insulating resin film, wherein the adhesive auxiliary layer having an adhesive auxiliary layer contains an epoxy resin (a), a cyanate resin (b), and an inorganic filer (c); and a printed circuit board obtained using the interlayer insulating resin film or the interlayer insulating resin film having an adhesive auxiliary layer.

### TECHNICAL FIELD

The present invention relates to an interlayer insulating resin film, an interlayer insulating resin film having an adhesive auxiliary layer, and a printed circuit board.

### BACKGROUND

Recently, size reduction, weight saving, and multifunctionality of electronic devices have been increasingly developed, and in association therewith, high integration of LSI (Large Scale Integration), chip components, etc. has been developed, with the formation thereof rapidly changing into multiple pins and size reduction. Therefore, in order to improve the mounting density of electronic components, microwiring of multilayered printed circuit boards has been developed. As a manufacturing method of multilayered printed circuit boards corresponding to these needs, a multilayered printed circuit board having a buildup structure using an insulating resin film not including glass cloth in place of prepreg as an interlayer insulating resin film (hereinafter, also referred to as a “buildup layer”) is becoming popular as a printed circuit board suitable for weight saving, size reduction, and miniaturization.

In order to improve the processed measurement stability and decrease the amount of warpage after being mounted on a semiconductor, low thermal expansion is required for the buildup layer. As the main method for carrying out low thermal expansion of the buildup layer, a method of high filling a silica filler is considered. For example, low thermal expansion of the buildup layer is carried out by using not less than 40% by weight of the buildup layer as the silica filler (Patent documents 1 to 3).

On the other hand, computers and information communication units have been increasingly achieving technical advancements as well as increased functionality in recent years, with signals showing a tendency towards higher frequencies for processing large amounts of data at high speeds. In particular, the high frequency domain of GHz bands is used as the frequency domain of radio waves used for cellular phones and satellite broadcasts. Therefore, as an organic material used in high frequency domains, a material having a low relative permittivity and dielectric tangent has been desired in order to prevent transmission loss due to high frequency.

The ability to form an interlayer insulating resin film with a resin composition containing a cyanate resin and having exceptional dielectric characteristics as a resin composition used for an interlayer insulating resin film of multilayered printed circuit boards is known. However, the adhesion strength between the interlayer insulating resin film obtained from a resin composition containing a cyanate resin and a circuit board after accelerated environment testing is not necessarily satisfactory.

### PRIOR ART DOCUMENTS

### Patent Documents

Patent document 1: JP 2007-87982 A

Patent document 2: JP 2009-280758 A

Patent document 3: JP 2005-39247 A

### SUMMARY OF THE INVENTION

### Problem to be Solved by the Invention

The present invention has been created in light of the above described problems, with an object of providing an interlayer insulating resin film, an interlayer insulating resin film having an adhesive auxiliary layer, and a printed circuit board obtained using the interlayer insulating resin film or the interlayer insulating resin film having an adhesive auxiliary layer, with which it is possible to obtain an interlayer insulating layer having exceptional adhesion to a circuit board even after accelerated environmental testing, along with exceptional heat resistance, dielectric characteristics, and low thermal expansion.

### Means for Solving the Problems

Upon investigating the abovementioned problems, the inventors of the present invention found that the present invention could be used to resolve such problems. Specifically, the present invention may provide the following [1] to [7].

[1] An interlayer insulating resin film containing epoxy resin (A), cyanate resin (B), and dicyandiamide (C).

[2] The interlayer insulating resin film according to the abovementioned [1], further containing inorganic filler (D).

[3] The interlayer insulating resin film according to the abovementioned [2], wherein inorganic filler (D) is silica.

[4] The interlayer insulating resin film according to any one of the abovementioned [1] to [3], wherein the contained amount of dicyandiamide (C) is 0.005 to 5.0 parts by mass with respect to the total solid content conversion of 100 parts by mass of epoxy resin (A) and cyanate resin (B).

[5] An interlayer insulating resin film including an adhesive auxiliary layer, with an adhesive auxiliary layer provided on one surface of the interlayer insulating resin film according to any one of the abovementioned [1] to [4], wherein the adhesive auxiliary layer contains epoxy resin (a), cyanate resin (b), and inorganic filler (c).

[6] The interlayer insulating resin film including an adhesive auxiliary layer according to the abovementioned [5], further including a support body provided on the opposite surface from the surface on which the interlayer insulating resin film of the adhesive auxiliary layer is provided.

[7] A printed circuit board, including the interlayer insulating resin film according to any one of the abovementioned [1] to [4], or the interlayer insulating resin film having an adhesive auxiliary layer according to the abovementioned [5] or [6].

### Effect of the Invention

According to the present invention, it is possible to provide an interlayer insulating resin film, an interlayer insulating resin film having an adhesive auxiliary layer, and a printed circuit board obtained using the interlayer insulating resin film or the interlayer insulating resin film having an adhesive auxiliary layer, with which it is possible to obtain an interlayer insulating layer having exceptional adhesion to a circuit board even after accelerated environmental testing, along with exceptional heat resistance, dielectric characteristics, and low thermal expansion.

### MODE FOR CARRYING OUT THE INVENTION

[Interlayer Insulating Resin Film]

The interlayer insulating resin film of the present invention contains epoxy resin (A), cyanate resin (B), and dicyandiamide (C).

<Epoxy Resin (A)>

Epoxy resin (A) is not particularly limited; however, for example, bisphenol A epoxy resin, bisphenol F epoxy resin, bisphenol S epoxy resin, phenol novolak epoxy resin, cresol novolak epoxy resin, biphenyl epoxy resin, aralkyl type epoxy resin, naphthol-type epoxy resin, anthracene type epoxy resin, dicyclopentadiene type epoxy resin, naphthalene type epoxy resin, fluorene type epoxy resin, xanthene type epoxy resin, etc. can be considered. These epoxy resins (A) may be used on their own or two or more different epoxy resins may be combined.

In terms of heat resistance, insulation reliability, and adhesion to a circuit board, epoxy resin (A) may be novolak type epoxy resin, bisphenol F epoxy resin, naphthalene type epoxy resin, aralkyl type epoxy resin, naphthalene type epoxy resin, and aralkyl type epoxy resin. Further, naphthalene type epoxy resin may be used in conjunction with aralkyl type epoxy resin. As an aralkyl type epoxy resin, an aralkyl type epoxy resin represented by the following general formula (1) may be used.

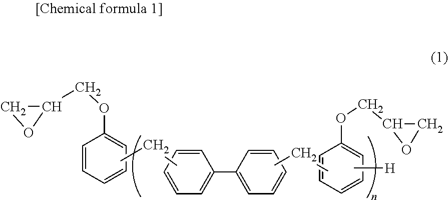

(n denotes the numbers 1 to 10.)

Commercial products may be used as epoxy resin (A). As commercial products, for example, N-740 (epoxy equivalent 180), N-770 (epoxy equivalent 188), N-673 (epoxy equivalent 211), N-830S (epoxy equivalent 168) (the above is manufactured by DIC CORPORATION, trade name), NC-7000L (epoxy equivalent 231), NC-3000H (epoxy equivalent 289), NC-3000L, NC-3000, NC-3100, NC-2000L (epoxy equivalent 237) (the above is manufactured by Nippon Kayaku Co., Ltd., trade name), etc. can be considered.

The contained amount of epoxy resin (A) in the interlayer insulating resin film is not particularly limited; however, it is preferably 5 to 30 parts by mass, and more preferably 10 to 25 parts by mass, with respect to a solid content of 100 parts by mass contained in the interlayer insulating resin film.

When the contained amount of epoxy resin (A) is not less than 5 parts by mass with respect to a solid content of 100 parts by mass contained in the interlayer insulating resin film, adhesion to the conductor layer tends to be improved, whereas when it is not more than 30 parts by mass, there is a tendency to be able to decrease the dielectric tangent since the contained amount of cyanate resin (B) can be sufficiently maintained.

In the present specification, the solid content contained in the interlayer insulating resin film means the residue obtained by eliminating volatile components from the interlayer insulating resin film.

### <Cyanate Resin (B)>

Cyanate resin (B) is not particularly limited; however, for example, a bisphenol cyanate resin such as bisphenol A, bisphenol F, or bisphenol S, a novolak phenol cyanate resin such as phenol novolak or alky phenol novolak, a dicyclopentadiene type cyanate resin, and partly triazinated prepolymers, etc. are considered. These cyanate resins (B) may be used on their own or two or more different ones may be combined. Among these cyanate resins, cyanate resin (B) may be bisphenol A cyanate resin or a prepolymer of bisphenol A cyanate resin.

The weight average molecular weight of cyanate resin (B) is not particularly limited; however, it is preferably 200 to 4500, and more preferably 300 to 3000.

When the weight average molecular weight is not less than 200, crystallization of cyanate resin (B) is inhibited, with the solubility of organic solvents tending to be good. Moreover, if the weight average molecular weight is not more than 4500, increased viscosity is inhibited, with operability tending to be good.

The weight average molecular weight is measured using the standard curve of standard polystyrene by gel permeation chromatography (GPC) (manufactured by TOSO corporation).

The contained amount of cyanate resin (B) in the interlayer insulating resin film is not particularly limited; however, it is preferably 2 to 50 parts by mass, more preferably 4 to 40 parts by mass, further preferably 5 to 30 parts by mass, and still further preferably 5 to 20 parts by mass, with respect to a solid content of 100 parts by mass contained in the interlayer insulating resin film. When the contained amount of cyanate resin (B) is not less than 2 parts by mass with respect to a solid content of 100 parts by mass contained in the interlayer insulating resin film, good dielectric characteristics, good heat resistance, and low thermal expansion tend to be obtained, while when it is not more than 50 parts by mass, good adhesion to a circuit board after accelerated environmental testing tends to be obtained.

### <Dicyandiamide (C)>

The contained amount of dicyandiamide (C) in the interlayer insulating resin film is not particularly limited; however, in terms of preventing the lowering of adhesion to the circuit board after accelerated environmental testing, it is preferably not less than 0.005 parts by mass, more preferably not less than 0.01 parts by mass, further preferably not less than 0.03 parts by mass, still further preferably not less than 0.25 parts by mass, and even further preferably not less than 0.5 parts by mass, with respect to the total solid content conversion of 100 parts by mass of epoxy resin (A) and cyanate resin (B). Moreover, in terms of preventing aggregates of dicyandiamide (C) from being deposited on the film coating as well as deterioration of dielectric characteristics, the upper limit value of the contained amount of dicyandiamide (C) is preferably not more than 5.0 parts by mass, more preferably not more than 3.0 parts by mass, and further preferably not more than 1.5 parts by mass, with respect to the total solid content conversion of 100 parts by mass of epoxy resin (A) and cyanate resin (B).

Moreover, in the contained amount of dicyandiamide (C) in the interlayer insulating resin film, dicyandiamide (C) equivalent [(blending quantity of dicyandiamide (C)/active hydrogen equivalent of dicyandiamide (C))/(blending quantity of epoxy resin (A)/epoxy equivalent of epoxy resin (A))] is preferably 0.005 to 0.5, more preferably 0.04 to 0.3, and further preferably 0.08 to 0.13, with respect to epoxy resin (A). When the equivalent is not less than 0.005, adhesion to the circuit board after accelerated environmental testing tends to be good, while when it is not more than 0.5, dielectric characteristics thereof tend to be good.

### <Inorganic Filler (D)>

The interlayer insulating resin film of the present invention may further include inorganic filler (D). Thereby, low thermal expansion of the interlayer insulating resin film is attained.

The additive amount in the case of adding inorganic filler (D) differs depending on the properties and functions of the interlayer insulating resin film of the present invention; however, for example, it is preferably 50 to 500 parts by mass, more preferably 100 to 400 parts by mass, and further preferably 150 to 300 parts by mass, with respect to a solid content conversion of 100 parts by mass of the resin component in the interlayer insulating resin film.

The term “resin component” means other thermosetting resins and thermoplastic resins that may be added as epoxy resin (A), cyanate resin (B), dicyandiamide (C), and other components to be mentioned later.

As inorganic filler (D), silica, alumina, barium sulfate, talc, clay, mica powder, aluminum hydroxide, magnesium hydroxide, calcium carbonate, magnesium carbonate, magnesium oxide, boron nitride, aluminum borate, barium titanate, strontium titanate, calcium titanate, magnesium titanate, bismuth titanate, oxidized titanium, barium zirconate, calcium zirconate, etc. are considered. Among these, silica is preferable. These inorganic fillers may be used on their own or two or more different ones may be combined.

Moreover, the average grain diameter of the inorganic filler (D) is preferably not more than 5 μm. If the average grain diameter is not more than 5 μm, when a circuit pattern is formed on the interlayer insulating resin film, a fine pattern tends to be able to be stably formed. The average grain diameter means the grain diameter of the point equivalent to 50% volume when a cumulative frequency distribution curve of the grain diameter is plotted, wherein the overall volume of the grain is 100% and the average grain diameter can be measured by a particle counter, etc. using a laser diffraction scattering method.

Moreover, a surface preparation may be applied to inorganic filler (D) by a surface preparation agent such as a silane coupling agent in order to improve moisture resistance.

The surface preparation agent is not particularly limited; however, it is preferably an aminosilane coupling agent, more preferably a silicon oligomer coupling agent in terms of the embedding properties between wires as well as the flatness after lamination and thermal curing. Specifically, inorganic filler (D) is preferably an inorganic filler applied with a surface preparation using an aminosilane coupling agent, more preferably an inorganic filler applied with a surface preparation using a silicon oligomer coupling agent. Moreover, as inorganic filler (D), an inorganic filler with a surface preparation applied using an aminosilane coupling agent is preferably used in conjunction with an inorganic filler with a surface preparation applied using a silicon oligomer coupling agent, and the combination ratio thereof is preferably a ratio in which the contained amount of inorganic filler applied with a surface preparation using an aminosilane coupling agent is preferably 60 to 90 parts by mass, more preferably 70 to 80 parts by mass with respect to 100 parts by mass of inorganic filler (D).

### <Other Components>

Further, as the interlayer insulating resin film of the present invention, besides each of the abovementioned components, without inhibiting the effect of the present invention, as necessary, other thermosetting resins, other thermoplastic resins, and addition agents such as fire retardants, antioxidants, fluidity modifiers, and hardening accelerators can be used.

The interlayer insulating resin film of the present invention may be any one surface on which a support body is provided.

As a support body, a polyolefin film such as polyethylene, polypropylene, and polyvinyl chloride, polyethyleneterephthalate (hereinafter, also referred to as “PET”), a polyester film such as polyethylene naphthalate, and various plastic films such as polycarbonate film and polyimide film may be considered. Moreover, metallic foils such as exfoliate paper, copper foil, and aluminum foil may be used. A support body and protection film to be mentioned later may undergo a surface preparation such as mat treatment or corona treatment. Moreover, they may undergo a mold release treatment with a silicone resin release agent, alkyd resin release agent, fluororesin release agent, etc. The thickness of the support body is not particularly limited; however, it is preferably 10 to 150 μm, more preferably 25 to 50 μm.

The usage of the interlayer insulating resin film of the present invention is not particularly limited; however, it may be used for purposes requiring an interlayer insulating resin film such as a glue film, an insulating resin sheet such as prepreg, a circuit board, a solder mask, an underfill material, a die bonding material, a semiconductor sealing material, a resin for filling a hole, a resin for filling parts, etc. Among these, it can be preferably used to form an interlayer insulating resin film upon manufacturing a multilayered printed circuit board.

Subsequently, the manufacturing method of the interlayer insulating resin film of the present invention will be described.

### <Manufacturing Method of the Interlayer Insulating Resin Film>

The interlayer insulating resin film of the present invention can be manufactured, for example, as follows.

Upon manufacturing the interlayer insulating resin film, first, epoxy resin (A), cyanate resin (B), dicyandiamide (C)), and other components to be used as necessary are preferably made into a resin varnish dissolved or dispersed in an organic solvent (hereinafter, also referred to as a “varnish for an interlayer insulating resin film”).

The varnish for an interlayer insulating resin film can be manufactured according to a method involving blending epoxy resin (A), cyanate resin (B), dicyandiamide (C), and other components with an organic solvent, then mixing them using a known agitator, etc.

As the organic solvent, for example, ketones such as acetone, methyl ethyl ketone, methyl isobutyl ketone, and cyclohexanone, acetoacetic acid esters such as ethyl acetate, butyl acetate, cello solve acetate, propylene glycol monomethyl ether acetate, and carbitol acetate, carbitols such as cello solve and butyl carbitol, aromatic hydrocarbons such as toluene and xylene, amide type solvents such as dimethylformamide, dimethylacetamide, and N-methylpyrrolidone, etc. can be considered. These organic solvents may be used on their own or two or more different ones may be combined.

The blending quantity of the organic solvent is preferably 10 to 50 parts by mass, and preferably 10 to 35 parts by mass, with respect to 100 parts by mass of varnish for an interlayer insulating resin film.

It is possible to obtain an interlayer insulating resin film by thermally drying the thus manufactured varnish for an interlayer insulating resin film after applying it to a support body.

The support body is not particularly limited; however, for example, the same one as the support body provided in the abovementioned interlayer insulating resin film of the present invention can be considered.

As a method of applying the varnish for the interlayer insulating resin film to a support body, for example, it is possible to use a coating machine known to persons skilled in the art such as a comma coater, a bar coater, a kiss coater, a roll coater, a gravure coater, and a die coater. These coating machines may be appropriately selected in accordance with the film thickness.

The drying temperature and drying time may be appropriately determined in accordance with the usage amount of the organic solvent, the boiling temperature of the organic solvent to be used, etc.; however, for example, in the case of a varnish for an interlayer insulating resin film containing 30 to 60% by weight of an organic solvent, the interlayer insulating resin film is preferably formed by drying this varnish at 50 to 150° C. for 3 to 10 minutes.

The contained amount of volatile components (mainly, organic solvents) in the interlayer insulating resin film of the present invention is preferably not more than 10% by weight, and more preferably not more than 5% by weight.

The thickness of the interlayer insulating resin film of the present invention may be appropriately determined in accordance with the required performance; however, the thickness thereof may be determined to be not less than the thickness of the conductor layer of the conductor layer of the circuit board on which the interlayer insulating resin film of the present invention is layered. Specifically, the thickness of the interlayer insulating resin film is preferably 10 to 100 μm since the thickness of the conductor layer placed on the circuit board is preferably within the range of 5 to 70 μm.

A protection film may be layered on the surface of the interlayer insulating resin film opposite the surface formed on the support body. The thickness of the protection film is not particularly limited but, for example, may be 1 to 40 μm. Layering the protection film makes it possible to prevent dust from sticking to the surface of the interlayer insulating resin film and prevent the surface from being scratched. The interlayer insulating resin film can also be stored by being wound into a roll.

### [Interlayer Insulating Resin Film Including an Adhesive Auxiliary Layer]

The interlayer insulating resin film including an adhesive auxiliary layer of the present invention includes one surface of the interlayer insulating resin film of the abovementioned present invention on which an adhesive auxiliary layer is provided.

The adhesive auxiliary layer is placed between the interlayer insulating resin film forming the interlayer insulating resin film of the present invention and a conductor layer formed by plating, which is provided in order to improve adhesion to the conductor layer. In terms of forming fine wiring, an adhesive auxiliary layer is preferable since a smooth surface can be obtained by providing an adhesive auxiliary layer, giving good adhesion strength to the conductor layer formed by plating. As the adhesive auxiliary layer, one capable of giving good adhesiveness to the conductor layer formed by plating is preferable. As an example thereof, one containing epoxy resin (a), cyanate resin (b), and inorganic filler (c) is considered.

<Epoxy Resin (a)>

Epoxy resin (a) is not particularly limited, with the same one as the abovementioned epoxy resin (A) considered.

Among these epoxy resins, in terms of adhesion to the conductor layer, an alkyl phenol novolak epoxy resin is preferable, while in terms of lowering the thermal expansion rate of the obtained interlayer insulating resin film, a naphthalene cresol novolak epoxy resin is preferable.

The contained amount of epoxy resin (a) in the adhesive auxiliary layer is not particularly limited; however, it is preferably 40 to 90 parts by mass, more preferably 45 to 70 parts by mass, and further preferably 50 to 60 parts by mass, with respect to a solid content of 100 parts by mass contained in the adhesive auxiliary layer. If the contained amount of epoxy resin (a) is not less than 40 parts by mass, there is a tendency for the printed circuit board to be moisture resistant, with excellent adhesion between the conductor layer and the interlayer insulating resin film.

Further, according to the present specifications, the solid content contained in the adhesive auxiliary layer means residue obtained by eliminating volatile components from the interlayer insulating resin film.

<Cyanate Resin (b)>

Cyanate resin (b) is not particularly limited; however, the same one as the abovementioned cyanate resin (B) is considered, with the same weight average molecular weight.

The contained amount of cyanate resin (b) in the adhesive auxiliary layer is not particularly limited; however, it is preferably 20 to 60 parts by mass, more preferably 30 to 50 parts by mass, and further preferably 35 to 45 parts by mass, with respect to a solid content of 100 parts by mass contained in the adhesive auxiliary layer. If the contained amount of cyanate resin (b) is not less than 20 parts by mass with respect to a solid content of 100 parts by mass contained in the adhesive auxiliary layer, good dielectric characteristics, good heat resistance, and good low thermal expansion tend to be obtained, while if it is not more than 60 parts by mass, adhesion to the conductor layer after accelerated environmental testing tends to be good.

<Inorganic Filler (c)>

Upon laser machining, scattering of the resin is prevented by blending inorganic filler (c) into the adhesive auxiliary layer, making it possible to adjust the laser machining shape of the interlayer insulating resin film formed by the interlayer insulating resin film including an adhesive auxiliary layer. Moreover, upon roughening the surface of the interlayer insulating resin film formed on the interlayer insulating resin film including an adhesive auxiliary layer, a moderate roughened surface is formed, making it possible to express good adhesion strength on a conductor layer formed by plating.

As inorganic filler (c), the same inorganic fillers considered for the abovementioned inorganic filler (D) are considered. Among these, silica is preferred. Moreover, as the silica, fumed silica, colloidal silica, etc. are considered.

In terms of forming fine wiring on the interlayer insulating resin film formed by the adhesive auxiliary layer, the specific surface area of inorganic filler (c) is preferably not less than 20 m²/g, and more preferably not less than 50 m²/g. The upper limit value of the specific surface area of inorganic filler (c) is not limited; however, in terms of ease of acquisition, it is preferably not more than 500 m²/g, and more preferably not more than 200 m²/g.

The specific surface area can be obtained via the BET method due to the low temperature and low humidity physical absorption of inert gases. Specifically, molecules for which the absorption occupation area is known are absorbed on the surface of the powder particles by liquid nitrogen, making it possible to obtain the specific surface area of the power particles from the absorption amount.

Commercial products with a specific surface area not less than 20 m²/g are preferably used as inorganic filler (c). As commercial products, for example, AEROSIL R972 (manufactured by NIPPON AEROSIL CO., LTD., trade name, specific surface area 110 m²/g) that is a fumed silica, AEROSIL R202 (manufactured by NIPPON AEROSIL CO., LTD., trade name, specific surface area 100 m²/g) that is a fumed silica, PL-1 (manufactured by FUSO CHEMICAL CO., LTD., trade name, specific surface area 181 m²/g) colloidal silica, PL-7 (manufactured by FUSO CHEMICAL CO., LTD., trade name, specific surface area 36 m²/g) colloidal silica, etc. are considered. Moreover, in terms of improving the moisture resistance, it is preferably an inorganic filler applied with a surface preparation by a surface preparation agent such as a silane coupling agent.

The contained amount of inorganic filler (c) in the adhesive auxiliary layer is preferably 3 to 30 parts by mass, more preferably 3 to 25 parts by mass, and further preferably 5 to 20 parts by mass, with respect to a solid content conversion of 100 parts by mass of a resin component in the adhesive auxiliary layer. If the contained amount of inorganic filler (c) is not less than 3 parts by mass with respect to a solid content conversion of 100 parts by mass of a resin component in the adhesive auxiliary layer, good laser machinability tends to be obtained, while if it is not more than 30 parts by mass, upon forming a conductor layer by plating after roughening the interlayer insulating resin film, sufficient adhesion force between the adhesive auxiliary layer and the conductor layer tends to be obtained.

### <Other Components>

Regarding the adhesive auxiliary layer, besides each of the abovementioned components, without inhibiting the effect of the present invention, as necessary, other thermosetting resins, other thermoplastic resins, and addition agents such as fire retardants, antioxidants, fluidity modifiers, and hardening accelerators can be used.

The interlayer insulating resin film having an adhesive auxiliary layer may be further provided with a support body on the surface opposite the surface on which the interlayer insulating resin film of the adhesive auxiliary layer is provided.

As a support body, the same support body used in the manufacturing method of the interlayer insulating resin film of the present invention is considered.

### <Manufacturing Method of an Interlayer Insulating Resin Film Including an Adhesive Auxiliary Layer>

The interlayer insulating resin film including an adhesive auxiliary layer of the present invention can be manufactured by, for example, a method of forming an adhesive auxiliary layer on the support body, then forming an interlayer insulating resin film on the adhesive auxiliary layer.

Upon manufacturing the adhesive auxiliary layer, epoxy resin (a), cyanate resin (b), inorganic filler (c), and other components may be made into a resin varnish dissolved or dispersed in an organic solvent (hereinafter, also referred to as a “varnish for the adhesive auxiliary layer”).

The manufacturing method of the varnish for the adhesive auxiliary layer and the organic solvent used for manufacturing the varnish for the adhesive auxiliary layer are the same as the abovementioned varnish for the interlayer insulating resin film.

The blending quantity of the organic solvent is preferably 60 to 95 parts by mass, and more preferably 70 to 90 parts by mass, with respect to 100 parts by mass of the varnish for the adhesive auxiliary layer.

It is possible to form an interlayer insulating resin film having an adhesive auxiliary layer by thermally drying the thus manufactured varnish for the adhesive auxiliary layer after it is applied on a support body, and further, thermally drying the varnish for the interlayer insulating resin film after being applied thereon.

The varnish for the adhesive auxiliary layer and the method of applying the varnish for the interlayer insulating resin film, as well as the drying conditions after applying these varnishes, are the same as the application method and drying conditions in the manufacturing method of the interlayer insulating resin film of the present invention.

The thickness of the interlayer insulating resin film formed on the interlayer insulating resin film having an adhesive auxiliary layer of the present invention may be appropriately determined in accordance with the required performance; however, the thickness thereof is preferably determined to be not less than the thickness of the conductor layer of the conductor layer of the circuit board on which the interlayer insulating resin film is layered. Specifically, the thickness of the interlayer insulating resin film is preferably 10 to 100 μm since the thickness of the conductor layer placed on the circuit board usually is within the range of 5 to 70 μm. Moreover, the thickness of the adhesive auxiliary layer is not particularly limited; however, for example, 1 to 15 μm is preferable.

It is possible to further layer a protection film on the side of the interlayer insulating resin film having an adhesive auxiliary layer with no adhesive auxiliary layer provided. The thickness of the protection film is not particularly limited; however, for example, 1 to 40 μm is preferable. Layering the protection film makes it possible to prevent dust from sticking to the surface of the interlayer insulating resin film and prevent the surface from being scratched. The interlayer insulating resin film can also be stored by being wound into a roll.

### [Printed Circuit Board]

The printed circuit board of the present invention includes the interlayer insulating resin film of the present invention or the interlayer insulating resin film including an adhesive auxiliary layer.

A method of manufacturing a printed circuit board by laminating the interlayer insulating resin film of the present invention or the interlayer insulating resin film including an adhesive auxiliary layer on a circuit board will be described below.

The printed circuit board can be manufactured according to a manufacturing method including the following steps (1) to (5), wherein the support body may be separated or removed after step (1), step (2), or step (3).

Step (1): laminating the interlayer insulating resin film or the interlayer insulating resin film including an adhesive auxiliary layer of the present invention on one side or both sides of a circuit board.

Step (2): forming an interlayer insulating resin film by thermally setting the laminated interlayer insulating resin film or the laminated interlayer insulating resin film including an adhesive auxiliary layer.

Step (3): boring a circuit board with the interlayer insulating resin film formed.

Step (4): conducting roughening treatment on the surface of the interlayer insulating resin film.

Step (5): forming a conductor layer on the surface of the roughened interlayer insulating resin film by plating.

### <Step (1)>

Step (1) involves laminating the interlayer insulating resin film or the interlayer insulating resin film including an adhesive auxiliary layer of the present invention on one side or both sides of a circuit board. As the apparatus for laminating the interlayer insulating resin film or the adhesive auxiliary layer including the interlayer insulating resin film, a vacuum laminator is preferable. Commercial products may be used as the vacuum laminator. As a commercially available vacuum laminator, for example, a vacuum applicator manufactured by Nichigo-Morton Co., Ltd., a vacuum-pressing laminator manufactured by MEIKI CO., LTD., a roll-type dry coater manufactured by Hitachi Industries Co., Ltd., a vacuum laminator manufactured by Hitachi AIC Inc., etc. are considered.

Upon lamination, if the interlayer insulating resin film or the interlayer insulating resin film including an adhesive auxiliary layer has a protection film, the protection film is removed, after which, the interlayer insulating resin film or the interlayer insulating resin film including an adhesive auxiliary layer is pressure bonded to the circuit board while being pressed and heated.

When the interlayer insulating resin film including an adhesive auxiliary layer is used, the side of the interlayer insulating resin film with no adhesive auxiliary layer provided is arranged so as to oppose the side with the circuit of the circuit board formed.

The lamination conditions are not particularly limited; however, the interlayer insulating resin film or the interlayer insulating resin film having an adhesive auxiliary layer, and the circuit board are preheated as necessary, after which they may be laminated under reduced pressure at a pressure-bonding temperature (lamination temperature) of 60 to 140° C. a pressure-bonding pressure of 0.1 to 1.1 MPa (9.8×10⁴ to 107.9×10⁴ N/m²), and air pressure of 20 mmHg (26.7 hPa) or less. Moreover, the lamination method may be a batch type or a continuous type with a roll.

### <Step (2)>

Step (2) involves forming an interlayer insulating resin film by thermally setting the laminated interlayer insulating resin film or the laminated interlayer insulating resin film including an adhesive auxiliary layer, with each film laminated in step (1). In the present step, first, the circuit board on which the laminated interlayer insulating resin film or the laminated interlayer insulating resin film including an adhesive auxiliary layer is laminated in step (1) is cooled to approximately room temperature.

Subsequently, in the case of separating the support body, an interlayer insulating resin film is formed by heat setting the laminated interlayer insulating resin film or the laminated interlayer insulating resin film including an adhesive auxiliary layer, with each layer laminated on the circuit board after being separated. The heat setting conditions are not particularly limited; however, they are preferably selected to be within the range of, for example, 170 to 220° C. for 20 to 150 minutes. In the case of using a support body that has undergone mold release treatment, the support body may be separated after being thermally set.

In the case of manufacturing a printed circuit board with the laminated interlayer insulating resin film or the laminated interlayer insulating resin film including an adhesive auxiliary layer, hardened materials of the adhesive auxiliary layer and the interlayer insulating resin film are equivalent to the interlayer insulating resin film.

### <Step (3)>

Step (3) involves boring a circuit board with the interlayer insulating resin film formed. In this step, a via hole, through hole, etc. are formed by boring the interlayer insulating resin film and the circuit board formed in step (2) according to a method involving the use of a drill, laser, plasma, or a combination thereof, etc. A carbon dioxide laser, YAG laser, UV laser, excimer laser, etc. are generally used as the laser.

### <Step (4)>

Step (4) involves conducting roughening treatment on the surface of the interlayer insulating resin film. In this step, the surface of the interlayer insulating resin film formed in step (2) undergoes roughening treatment with an oxidant. At the same time, if a via hole, through hole, etc. are formed, “smears” generated upon forming these can be also removed.

The oxidant is not particularly limited; however, for example, ermanganic acid (potassium permanganate, sodium permanganate), bichromate, ozone, hydrogen peroxide, sulfuric acid, nitric acid, etc. are considered. Among these, the surface may be roughened and smears on the surface may be removed using an alcaline permanganic acid solution (for example, a potassium permanganate solution or a sodium permanganate solution), which is an oxidant generically used for roughening the interlayer insulating resin film upon manufacturing a printed circuit board according to the build up method.

### <Step (5)>

Step (5) involves forming a conductor layer on the surface of the roughened interlayer insulating resin film by plating. This step can use a semi-additive method of forming a power feeding layer on the surface of the interlayer insulating resin film by non-electrolytic plating, subsequently forming a plating resist that is the opposite pattern from a conductor layer, and forming a conductor layer (circuit) by electrolytic plating. Further, by undergoing annealing treatment at, for example, 150 to 200° C. for 20 to 90 minutes after forming a conductor layer, it is possible to further improve and stabilize the adhesion strength between the interlayer insulating resin film and the conductor layer.

Further, the present invention may include a step of roughening the surface of the conductor layer thus manufactured. Roughening the surface of the conductor layer has the effect of enhancing adhesion to the resin contacting the conductor layer. In order to roughen the conductor layer, Mech-Etch Bond CZ-8100, Mech-Etch Bond CZ-8101, Mech-Etch Bond CZ-5480 (these are trade names manufactured by MEC Co., Ltd.) etc., which are organic microetching agents, are preferably used.

### Examples

Hereinafter, the present invention will be specifically described with reference to examples; however, the present invention is not limited to these examples.

### [Synthesis of Prepolymer of Bisphenol a Dicyanate]

### Manufacturing Example 1

Toluene 269.6 g, 2,2-bis (4-cyanatophenyl) propane (manufactured by Lonza Japan, trade name: Primaset BADCY) 620.4 g, and ρ-(α-cumyl)phenol (manufactured by Tokyo Chemical Industry Co., Ltd.) 9.5 g were put into a separable flask with a capacity of 1 liter. Upon visually confirming that the 2,2-bis (4-cyanatophenyl) propane and ρ-(α-cumyl)phenol dissolved in the toluene, the liquid temperature was maintained at 100° C.; zinc naphthenate (manufactured by Wako Pure Chemical Industries, Ltd.) 0.46 g diluted in advance to 10% by weight with respect to a reaction solvent (in this review, toluene) as the reaction accelerator was then combined and reacted at 100° C. for three hours, yielding a prepolymer solution (solid content concentration of approximately 70% by weight) of bisphenol A dicyanate.

### [Manufacturing of an Interlayer Insulating Resin Film]

### Example 1

As inorganic filler (D), a silica filler (manufactured by Admatechs, trade name: SC-2050-KNK, a methyl isobutyl ketone dispersion liquid having a solid content concentration of 70% by weight) 51.2 parts by mass (solid content) which underwent aminosilane coupling agent treatment and silicafiller (manufactured by Admatechs, trade name: SC-2050-KC, a methyl isobutyl ketone dispersion liquid having a solid content concentration of 70% by weight) 17.1 parts by mass (solid content) which underwent silicon oligomer coupling agent (manufactured by Hitachi Chemical Co., Ltd., trade name: SC6000) treatment were blended.

Subsequently, phenoxy resin (manufactured by Mitsubishi Chemical Co., Ltd., trade name: YL7213B, methyl ethyl ketone solution having a solid content concentration of 35% by weight) 1.6 parts by mass (solid content), a dicyandiamide (manufactured by KANTO KAGAKU, Propylene glycol monomethyl ether solution having a solid content concentration of 0.8% by weight) 0.015 parts by mass (solid content), the prepolymer solution 8.4 parts by mass of bisphenol A dicyanate (solid content) obtained in Manufacturing Example 1, ρ-(α-cumyl)phenol (paracumyl phenol) (manufactured by Tokyo Chemical Industry Co., Ltd., molecular weight 212) 1.0 parts by mass, naphthalene type epoxy resin (manufactured by Nippon Kayaku Co., Ltd., trade name: NC-7000L, epoxy equivalent 231) 8.4 parts by mass, and an aralkyl type epoxy resin (manufactured by Nippon Kayaku Co., Ltd., trade name: NC-3000H, epoxy equivalent 289) 10.5 parts by mass were mixed in this order, then dissolved at room temperature using a high-speed rotary mixer.

Once dissolved, as a fire retardant, 1.7 parts by mass of 1,3-phenylenebis (di-2,6-xylenyl phosphate) (manufactured by DAIHACHI CHEMICAL INDUSTRY CO., LTD., trade name: PX-200), as an antioxidant, 0.08 parts by mass of 4,4′-butylidene bis-(6-t-butyl-3-methylphenol) (manufactured by Mitsubishi Chemical Co., Ltd., trade name: Yoshinomix BB), as a fluidity modifier, 0.08 parts by mass (solid content) of “BYK310” (manufactured by BYK Japan KK., trade name, xylene solution having a solid content concentration of 25% by weight), as an organic hardening accelerator, 0.02 parts by mass of 1-cyanoethyl-2-phenylimidazole (manufactured by SHIKOKU CHEMICALS CORPORATION, trade name: 2PZ-CN), and as a metallic hardening accelerator, 0.002 parts by mass of zinc naphthenate (manufactured by Wako Pure Chemical Industries, Ltd.) were blended, then stirred until dissolved. Next, they were dispersed by nanomizer treatment, yielding varnish 1 for manufacturing an interlayer insulating resin film.

Subsequently, this varnish 1 was applied on the PET film (38 μm thick), which was the support body, with a comma coater such that the thickness of the dried interlayer insulating resin film became 37 μm, after which it was dried at 105° C. for 2 minutes. Further, the amount of volatile components in the dried interlayer insulating resin film was 6% by weight. Subsequently, an interlayer insulating resin film having a support body and a protection film was obtained by adhering a polypropylene film 15 μm thick as a protection film onto the surface of the interlayer insulating resin film while rewinding it into a roll.

### Examples 2 to 5, Comparative Example 1

Varnishes 2 to 6 for manufacturing an interlayer insulating resin film were obtained in the same manner as Example 1 except that the blending quantity of dicyandiamide (manufactured by KANTO KAGAKU, Propylene glycol monomethyl ether solution having a solid content concentration of 0.8% by weight) in Example 1 was changed to the blending quantities shown in Table 1. Subsequently, an interlayer insulating resin film having a support body and a protection film was obtained using these varnishes 2 to 6 in the same manner as Example 1.

### [Manufacturing of an Interlayer Insulating Resin Film Including an Adhesive Auxiliary Layer]

### Example 6

The prepolymer solution of bisphenol A dicyanate 32.2 parts by mass (solid content) obtained in Manufacturing Example 1, naphthalenecresol novolak epoxy resin (manufactured by Nippon Kayaku Co., Ltd., trade name: NC-7000L, epoxy equivalent 231) 42.8 parts by mass, as an inorganic filler, silica filler-(manufactured by NIPPON AEROSIL CO., LTD., trade name: Aerosil R972, specific surface area 110 m2/g) 8.8 parts by mass, as an organic solvent, dimethylacetamide of 86.5 parts by mass with respect to all 100 parts by mass of the obtained varnish were blended, then stirred until the resin component had dissolved. Next, they were dispersed by nanomizer treatment, obtaining varnish 7 for manufacturing an adhesive auxiliary layer.

Subsequently, this varnish 7 was applied on the PET film (38 μm thick), which was a support body, with a comma coater such that the thickness of the dried adhesive auxiliary layer became 3 μm, after which it was dried at 140′C for 3 minutes to form an adhesive auxiliary layer on the PET film. Next, varnish 1 manufactured in Example 1 was applied on the abovementioned obtained adhesive auxiliary layer, after which it was applied with a comma coater such that the thickness of the dried interlayer insulating resin film became 40 μm, then dried at 140° C. for 2 minutes. Subsequently, an interlayer insulating resin film having an adhesive auxiliary layer having a support body and a protection film was obtained by adhering a polypropylene film 15 μm thick as a protection film onto the surface opposite the support body of the interlayer insulating resin film while rewinding it into a roll.

### Examples 7 to 10, Comparative Example 2

An interlayer insulating resin film with an adhesive auxiliary layer having a support body and a protection film was obtained in the same manner as Example 6 except that varnish 1 to be applied onto the adhesive auxiliary layer in Example 6 was changed into the varnish shown in Table 2.

### [Manufacturing of a Resin Plate]

The resin plate used to measure the glass transition temperature, the coefficient of thermal expansion, and the dielectric tangent was manufactured via the following procedure.

(I) The protection film was separated from the interlayer insulating resin film having a support body and the protection film obtained in Examples 1 to 5 and Comparative Example 1, after which it was dried at 110° C. for 10 minutes.

Subsequently, the interlayer insulating resin film having a dried support body was laminated on a gloss surface of copper foil (electric field copper foil, 12 μm thick) using a vacuum-pressing laminator (manufactured by MEIKI CO., LTD., trade name: MVLP-500/600-II) such that the interlayer insulating resin film came into contact with the copper foil, yielding layered body (1) with copper foil, an interlayer insulating resin film, and a support body, layered in this order. The lamination was carried out according to a method involving decompressing for 30 seconds, then pressing at 140° C. for 30 seconds, at a pressure-bonding pressure of 0.5 MPa Next, the support body was separated from layered body (1).

(II) Subsequently, the same interlayer insulating resin film having a support body and a protection film as the interlayer insulating resin film having a support body and a protection film used in the abovementioned (I) was prepared, and the same drying as in the abovementioned (I) was carried out after separating the protection film.

(III) Subsequently, layered body (1) with the support body obtained in the abovementioned (I) separated and an interlayer insulating resin film having the dried support body obtained in the abovementioned (II) was laminated such that the interlayer insulating resin films came into contact with each other under the same conditions as the abovementioned (I), yielding layered body (2) with a layer including copper foil, a layer made of two interlayer insulating resin films, and a support body, layered in this order. Next, the support body was separated from layered body (2).

(IV) Subsequently, layered body (2) with the support body obtained in the abovementioned (III) separated was layered onto an interlayer insulating resin film having the dried support body obtained by the same method as in the abovementioned (II) such that the interlayer insulating resin films came into contact with each to each other under the same conditions as the abovementioned (I), yielding layered body (3) with a layer including copper foil, a layer made of three interlayer insulating resin films, and a support body, layered in this order.

(V) Layered body (2) was manufactured according to the same method as in the abovementioned (I) to (III).

(VI) The support body of layered body (2) obtained in the abovementioned (V) and the support body of layered body (3) obtained in the abovementioned (I) to (IV) were respectively separated, the interlayer insulating resin films of layered body (2) and layered body (3) were adhered to each other, and press forming was carried out at a pressure-bonding pressure of 1.0 MPa at 175° C. for 60 minutes with a vacuum press. The obtained resin plate including copper foils on both surfaces was hardened at 190° C. for 2 hours, after which a resin plate approximately 0.2 mm thick was obtained by etching copper foil with ferric chloride.

### [Measuring Method of the Glass Transition Temperature]

The glass transition temperature was measured using a dynamic viscoelasticity measuring device (manufactured by UBM, trade name: DVE-V4). The resin plate manufactured as mentioned above was cut into pieces of 5 mm in width and 30 mm in length to be attached to a detector. The glass transition temperature was measured under the measurement conditions of a rate of temperature increase of 5° C./min, a frequency 10 Hz, and a measured temperature range of 40 to 350° C., with the temperature at which the loss elastic modulus becomes highest defined as the glass transition temperature. The results are shown in Table 1. This shows that the higher the glass transition temperature, the better the heat resistance.

### [Measuring Method of the Coefficient of Thermal Expansion]

The coefficient of thermal expansion was measured according to the tension weight method with a thermal mechanical analyzer (manufactured by TA Instruments, trade name: TMA2940). The resin plate manufactured as mentioned above was cut into pieces of 3 mm in width and 20 mm in length to be attached to a detector, then measured twice in a row under measurement conditions of a load of 0.05 N, a rate of temperature increase of 10° C./min, and a measurement temperature of −30 to 300° C. The average coefficient of thermal expansion (ppm) from 25° C. to 150° C. in the second measurement was calculated. The results are shown in Table 1. This shows that the lower the coefficient of thermal expansion, the better the low thermal expansion.

### [Measuring Method of the Dielectric Tangent]

The resin plate manufactured as mentioned above was cut into test pieces of 2 mm in width and 70 mm in length, then the dielectric tangent was measured with a network analyzer (manufactured by Agilent Technologies Japan, Ltd., trade name: E8364B) and a cavity resonator corresponding to 5 GHz. The measurement temperature was set to 25° C. The results are shown in Table 1. This shows that the lower the dielectric tangent, the better the dielectric characteristics.

### [Measuring Method of a Circuit Board and the Adhesion Strength Therewith]

Upon evaluating the adhesion strength to the circuit board, a substrate for evaluating the adhesion strength was manufactured according to the following procedures.

### (1) Surface Preparation of a Laminated Sheet

A substrate with the copper removed was obtained by etching both surfaces of a double sided copper clad laminated sheet (manufactured by Hitachi Chemical Co., Ltd., trade name: E-700GR, copper foil 12 μm thick, substrate 0.4 mm thick) with ammonium persulfate.

### (2) Surface Preparation of Copper Foil

A gloss surface of electrolytic copper foil (manufactured by Nippon Denkai, Ltd., trade name: YGP-35, 35 μm thick) was immersed in “Mech-Etch Bond CZ-8101” (trade name) manufactured by MEC Co., Ltd., and roughening treatment was carried out until the etching amount was 1 μm. In the present specifications, carrying out roughening treatment by immersing the gloss surface in “Mech-Etch Bond CZ-8101” (trade name) manufactured by MEC Co., Ltd. is referred to as “CZ treatment.”

### (3) Lamination of the Interlayer Insulating Resin Film

The protection film was separated from the interlayer insulating resin film having the support body and protection film manufactured in Examples 1 to 5 and Comparative Example 1. An interlayer insulating resin film having the obtained support body was laminated on the CZ treatment surface of the copper foil that underwent the CZ treatment in the abovementioned (2) with a batch type vacuum compressing laminator (manufactured by MEIKI CO., LTD.) such that the interlayer insulating resin film came into contact with the CZ treated surface. Lamination was carried out by a method involving laminating it at 100° C. for 30 seconds at a pressure-bonding pressure of 0.5 MPa after being decompressed for 30 seconds.

### (4) Hardening of the Interlayer Insulating Resin Film

After separating the support body from the interlayer insulating resin film laminated in the abovementioned (3), the interlayer insulating resin film was hardened at 190° C. for two hours with an explosion protection dryer, yielding a laminated sheet having an interlayer insulating resin film made by hardening the interlayer insulating resin film and copper layer as a conductor layer.

### (5) Press Forming

For the purpose of bonding it to the substrate obtained in the abovementioned (1), prepreg (manufactured by Hitachi Chemical Co., Ltd., trade name: E-679FG) and the laminated sheet obtained in the abovementioned (4) were laminated in the order of substrate, prepreg, interlayer insulating resin film, and copper layer, after which press forming was carried out at a pressure-bonding pressure of 1.5 MPa at 180° C. for 60 minutes with a vacuum-press to obtain a measurement substrate before a peel measuring part was manufactured.

### (6) Manufacturing of the Peel Measuring Part

A substrate for evaluating adhesion strength having a copper layer of 10 mm in width as the peel measuring part was obtained by forming a resist of 10 mm in width on the copper layer of the measurement substrate obtained in the abovementioned (5) and etching a copper layer with ferric chloride.

The adhesion strength between the interlayer insulating resin film and the copper layer was measured using the substrate for evaluating adhesion strength obtained as mentioned above according to the following method.

One end of the copper layer of the peel measuring part was peeled at the interface between the copper layer and the interlayer insulating resin film to be gripped by a gripper, then the load was measured while being vertically peeled at a pulling rate of 50 mm/minute at room temperature.

Moreover, after carrying out accelerated environmental testing for 100 hours on the same samples with a highly accelerated life apparatus (manufactured by ESPEC CORP.) under the conditions of 130° C., 85% RH, using the same method, the adhesion strength after accelerated environmental testing was measured. The maintenance rate (%) of the adhesion strength was calculated from the adhesion strength before and after accelerated environmental testing by the following formula, comparing the adhesion strengths before and after accelerated environmental testing. The results are shown in Table 1.

maintenance rate (%) of adhesion strength=(adhesion strength after accelerated environmental testing/adhesion strength before accelerated environmental testing)×100

### [Measuring Method of the Surface Roughness]

Upon measuring the surface roughness, a substrate for measuring surface roughness was created in the following order.

After cutting the interlayer insulating resin film having an adhesive auxiliary layer having the support body and protection film obtained in Examples 6 to 10 and Comparative Example 2 into pieces of 250 mm×250 mm in size, the protection film was separated.

The interlayer insulating resin film having an adhesive auxiliary layer having the obtained support body was laminated on a printed circuit board (manufactured by Hitachi Chemical Co., Ltd., trade name: E-700GR) that underwent CZ treatment using a vacuum-pressing laminator (manufactured by MEIKI CO., LTD., trade name: MVLP-500/600-II) such that the interlayer insulating resin film came into contact with the printed wiring board. Lamination was carried out according to a method involving laminating at 100° C. for 30 seconds at a pressure-bonding pressure of 0.5 MPa after being decompressed for 30 seconds.

Next, it was cooled to room temperature, after which the support body was separated and removed. Subsequently, after drying the printed circuit board with the interlayer insulating resin film having an adhesive auxiliary layer placed at 130° C. for 20 minutes, it was further hardened in an explosion protection dryer at 175° C. for 40 minutes, yielding a printed circuit board with an interlayer insulating resin film formed. Pieces obtained by cutting the printed circuit board to a size of 30 mm×40 mm were defined as test pieces.

The abovementioned obtained test pieces underwent immersing treatment in a sweller (manufactured by Rohm and Haas Electronic Materials K.K., trade name: CIRCUPOSIT MLB CONDITIONER211) heated to 80° C. for three minutes. Subsequently, the pieces underwent immersing treatment in a roughening liquid (manufactured by Rohm and Haas Electronic Materials K.K., trade name: CIRCUPOSIT MLB PROMOTER 213) heated to 80° C. for 8 minutes. Next, they underwent immersing treatment in a neutralizing solution (manufactured by Rohm and Haas Electronic Materials K.K., trade name: CIRCUPOSIT MLB NEUTRALIZER MLB216) heated to 45° C. for five minutes to be neutralized. In this way, the surface of the interlayer insulating resin film of the test pieces undergoing the roughening treatment was used as a substrate for measuring surface roughness.

The surface roughness of the substrate for measuring the surface roughness obtained as mentioned above was measured using a specific-contact type surface roughness meter (manufactured by Bruker AXS K.K., trade name: wykoNT9100), with an internal lens of 1 magnification and an external lens of 50 magnification, giving the arithmetic mean roughness (Ra). The results are shown in Table 2. Ra is preferably smaller from the aim of the present invention. Less than 200 nm is preferable in terms of fine wiring formation properties.

### [Measuring Method of the Adhesion Strength to Coated Copper]

Upon measuring the adhesion strength to coated copper, a substrate for measuring the adhesion strength to coated copper was created by the following procedures.

First, the substrate for measuring surface roughness was cut into pieces of 40 mm×60 mm, defined as test pieces.

The test pieces were treated with a 60° C. alkaline cleaner (manufactured by Atotech Japan K.K., trade name: cleaner security gantt 902) for five minutes to be degreased. After cleaning, they were treated for two minutes with a 23° C. predip liquid (manufactured by Atotech Japan K.K., trade name: predip neo gantt B). Next, they were treated for five minutes with a 40° C. activater liquid (manufactured by Atotech Japan K.K., trade name: activater neo gantt 834), allowing a palladium catalyst to be attached. Subsequently, they were treated for five minutes with a 30° C. reducing solution (manufactured by Atotech Japan K.K., trade name: reducer neo gantt WA).

The test pieces undergoing the abovementioned treatment were placed in a chemical copper liquid (manufactured by Atotech Japan K.K., trade name: basic print gantt MSK-DK), after which non-electrolytic plating was carried out until the plating thickness on the interlayer insulating resin film was approximately 0.5 μm. After non-electrolytic plating, the stress remaining in the plating film was alleviated and the test pieces underwent baking treatment at 120° C. for 15 minutes in order to remove the remaining hydrogen gas.

Subsequently, test pieces undergoing non-electrolytic plating underwent electrolytic plating, such that the plating the thickness on the interlayer insulating resin film further became 30 μm, forming a copper layer as a conductor layer. After electrolytic plating, the test pieces were hardened at 190′C for 90 minutes, yielding a measurement substrate before a peel measuring part was created.

A resist of 10 mm in width was formed on the copper layer of the obtained measurement substrate, yielding a substrate for measuring the adhesion strength to coated copper having a copper layer of 10 mm in width as the peel measuring part by etching the copper layer with ammonium persulfate.

A method for measuring the adhesion strength to coated copper was carried out in the same manner as the abovementioned measuring method of the adhesion strength to the circuit board. The results are shown in Table 2.

### [Reflow Heat Resistance]

Regarding the measurement of reflow heat resistance, a substrate for measuring reflow heat resistance was created by the following procedures.

A protection film was separated from the interlayer insulating resin film having an adhesive auxiliary layer having the support body and protection film obtained in Example 6 to 10 and Comparative Example 2. The interlayer insulating resin film having an adhesive auxiliary layer having the obtained support body was laminated on both surfaces of a printed circuit board having a conductor layer that underwent CZ treatment (manufactured by Hitachi Chemical Co., Ltd., trade name: MCL-E-679 (R), thickness 0.4 mm, 12 μm in copper thickness, provided with an internal layer circuit pattern) such that the interlayer insulating resin film came into contact with the conductor layer of the printed circuit board. Lamination was carried out according to a method involving pressing for 30 seconds, and then pressing at 100′C for 30 seconds at a pressure-bonding pressure of 0.5 MPa.

Next, it was cooled to room temperature, after which support bodies of both surfaces were separated and removed, yielding a printed circuit board with interlayer insulating resin films arranged on both surfaces. Subsequently, after drying the printed circuit board with interlayer insulating resin films arranged on both surfaces at 130° C. for 20 minutes, it was further hardened in an explosion protection dryer at 175′C for 40 minutes, yielding a printed circuit board with interlayer insulating resin films formed on both surfaces. Roughening treatment, non-electrolytic plating, and electrolytic plating were carried out on the obtained printed circuit board under the same conditions as the substrate in order to measure the adhesion strength to the coated copper. Next, post-curing was carried out at 190° C. for two hours, yielding a substrate for measuring reflow heat resistance.

This reflow heat resistance substrate for measurement was allowed to pass through a 265 Creflow furnace (manufactured by TAMURA Corporation, feeding rate of 0.61 m/min) and the number of passings until the generation of swelling (blister) was measured four times, with the average number of passings therethrough defined as the index of reflow heat resistance. The results are shown in Table 2. It is shown that the greater the average number of passings, the better the reflow heat resistance.

TABLE 1

| | | | Example | Example | Example | Example | Example | Comparative |
|---|---|---|---|---|---|---|---|---|
| | | | 1 | 2 | 3 | 4 | 5 | Example 1 |
| Varnish number | | | 1 | 2 | 3 | 4 | 5 | 6 |
| Blending | Epoxy resin (A) | NC-7000L | 8.4 | 8.4 | 8.4 | 8.4 | 8.4 | 8.4 |
| quantity | | NC-3000H | 10.5 | 10.5 | 10.5 | 10.5 | 10.5 | 10.5 |
| (parts by | Cynate resin (B) | Cyanate resin | 8.4 | 8.4 | 8.4 | 8.4 | 8.4 | 8.4 |
| mass) | | obtained in | | | | | | |
| | | Manufacturing | | | | | | |
| | | Example 1 | | | | | | |
| | Dicyandiamide (C) | | 0.015 | 0.046 | 0.076 | 0.152 | 0.301 | 0 |
| | Inorganic filler (D) | SC-2050-KMK | 51.2 | 51.2 | 51.2 | 51.2 | 51.2 | 51.2 |
| | | SC-2050-KC | 17.1 | 17.1 | 17.1 | 17.1 | 17.1 | 17.1 |
| | Other components | Paracumyl phenol | 1.0 | 1.0 | 1.0 | 1.0 | 1.0 | 1.0 |
| | | YL-7213B | 1.6 | 1.6 | 1.6 | 1.6 | 1.6 | 1.6 |
| | Fire retardant | PX-200 | 1.7 | 1.7 | 1.7 | 1.7 | 1.7 | 1.7 |
| | Antioxidant | Yoshinomix BB | 0.08 | 0.08 | 0.08 | 0.08 | 0.08 | 0.08 |
| | Fluidity modifier | BYK310 | 0.08 | 0.08 | 0.08 | 0.08 | 0.08 | 0.08 |
| | Organic hardening | 2PZ-CN | 0.02 | 0.02 | 0.02 | 0.02 | 0.02 | 0.02 |
| | accelerator | | | | | | | |
| | Metallic hardening | Zinc naphthenate | 0.002 | 0.002 | 0.002 | 0.002 | 0.002 | 0.002 |
| | accelerator | | | | | | | |
| Contained amount of | | (parts by mass)*1 | 0.05 | 0.17 | 0.28 | 0.56 | 1.10 | 0 |
| dicyandiamide (C) | | (equivalent)*2 | 0.01 | 0.03 | 0.05 | 0.10 | 0.20 | 0 |
| Evaluation | Heat resistance | Glass transition | 181 | 180 | 182 | 180 | 181 | 180 |
| results | | temperature (° C.) | | | | | | |
| | Low thermal | Coefficient of | 22.0 | 23.1 | 22.2 | 22.5 | 21.9 | 22.4 |
| | expansion | thermal expansion | | | | | | |
| | | (ppm) | | | | | | |
| | Dielectric | Dielectric tangent | 0.0072 | 0.0074 | 0.0077 | 0.0080 | 0.0084 | 0.0070 |
| | characteristics | | | | | | | |
| | Adhesion strength | Before | 0.53 | 0.54 | 0.48 | 0.46 | 0.40 | 0.48 |
| | to a circuit board | accelerated | | | | | | |
| | | environmental | | | | | | |
| | | testing (kgf/cm) | | | | | | |
| | | After accelerated | 0.13 | 0.17 | 0.25 | 0.29 | 0.21 | 0.05 |
| | | environmental | | | | | | |
| | | testing (kgf/cm) | | | | | | |
| | | Maintenance rate | 25 | 31 | 52 | 63 | 53 | 10 |
| | | (%) of adhesion | | | | | | |
| | | strength | | | | | | |

*1represents the contained amount of Epoxy resin (A) and cyanate resin (B) with respect to the total solid content conversion of 100 parts by mass.
*2represents the equivalent of dicyandiamide (C) with respect to epoxy resin (A), namely, [blending quantity of dicyandiamide (C)/active hydrogen equivalent of dicyandiamide (C))/(blending quantity of epoxy resin (A)/epoxy equivalent of epoxy resin (A))].

The details of the chemical compounds described in Table 1 are as follows.
NC-7000L: naphthalene type epoxy resin, manufactured by Nippon Kayaku Co., Ltd., trade name: NC-7000L, epoxy equivalent 231
NC-3000H: aralkyl type epoxy resin, manufactured by Nippon Kayaku Co., Ltd., trade name: NC-3000H, epoxy equivalent 289
Dicyandiamide: manufactured by KANTO KAGAKU
SC-2050-KNK: silica filler that underwent aminosilane coupling agent treatment silica filler, manufactured by Admatechs, trade name: SC-2050-KNK
SC-2050-KC: silica filler that underwent silicon oligomer coupling agent (manufactured by Hitachi Chemical Co., Ltd., trade name: SC6000) treatment manufactured by Admatechs, trade name: SC-2050-KC
Paracumyl phenol: ρ-(α-cumyl)phenol, manufactured by Tokyo Chemical Industry Co., Ltd., molecular weight 212
YL-7213B: phenoxy resin, manufactured by Mitsubishi Chemical Co., Ltd., trade name: YL7213B
PX-200: 1, 3-phenylenebis (di 2, 6-xylenyl phosphate), manufactured by DAIHACHI CHEMICAL INDUSTRY CO., LTD., trade name: PX-200
Yoshinomix BB: 4,4′-butylidene bis (6-t-butyl-methylphenol), manufactured by Mitsubishi Chemical Co., Ltd., trade name: Yoshinomix BB
BYK310: manufactured by BYK Japan KK., trade name: BYK310
2PZ-CN: 1-cyanoethyl2-phenylimidazole, manufactured by SHIKOKU CHEMICALS CORPORATION, trade name: 2PZ-CN
Zinc naphthenate: manufactured by Wako Pure Chemical Industries, Ltd.

From Table 1, it can be seen that Examples 1 to 5 better maintain the glass transition temperature, coefficient of thermal expansion, and dielectric tangent compared to Comparative Example 1. Moreover, it can also be seen that Examples 1 to 5 have excellent adhesion to copper foil even after accelerated environmental testing in the evaluation of the adhesion strength to a circuit board. Thus, it was found that, even when the interlayer insulating resin film of the present invention is layered on a circuit board to form an interlayer insulating resin film, the conductor layer (copper layer) and the interlayer insulating resin film of the circuit board have good adhesion strength even after accelerated environmental testing. Specifically, it was found that an interlayer insulating resin film excellent in adhesion to a circuit board, and further, excellent in low thermal expansion, heat resistance, and dielectric characteristics, is obtained by the interlayer insulating resin film of the present invention.

TABLE 2

| | | | | | | | Comparative |
|---|---|---|---|---|---|---|---|
| | | Example 6 | Example 7 | Example 8 | Example 9 | Example 10 | Example 2 |
| Number of the varnish used for forming | | 1 | 2 | 3 | 4 | 5 | 6 |
| the interlayer insulating resin film | | | | | | | |
| Evaluation | Surface roughness (Ra) | 183 | 190 | 194 | 192 | 185 | 180 |
| results | (nm) | | | | | | |
| | Adhesion strength | 1.0 | 0.9 | 1.0 | 1.0 | 1.0 | 1.0 |
| | (kgf/cm) to coated copper | | | | | | |
| | Reflow heat resistance | 10 | 15 | 18 | 20 | 21 | 5 |
| | (number of times) | | | | | | |

From Table 2, it can be seen that, compared to Comparative Example 2, Examples 6 to 10 employing the interlayer insulating resin film having an adhesive auxiliary layer of the present invention can yield an interlayer insulating resin film excellent in reflow heat resistance while maintaining surface roughness and adhesion strength to coated copper.

### INDUSTRIAL APPLICABILITY

The interlayer insulating resin film of the present invention can provide an interlayer insulating resin film excellent in low thermal expansion, heat resistance, and dielectric characteristics, particularly with little degradation of adhesion to a circuit board even after accelerated environmental testing. Accordingly, the interlayer insulating resin film of the present invention is useful for electrical products such as computers, cellular phones, digital cameras, and TVs, along with vehicles such as motorcycles, cars, trains, ships, and airplanes.

1. An interlayer insulating resin film comprising epoxy resin (A), cyanate resin (B), and dicyandiamide (C).

2. The interlayer insulating resin film according to claim 1, further comprising inorganic filler (D).

3. The interlayer insulating resin film according to claim 2, wherein inorganic filler (D) is silica.

4. The interlayer insulating resin film according to claim 1, wherein the contained amount of dicyandiamide (C) is 0.005 to 5.0 parts by mass with respect to the total solid content conversion of 100 parts by mass of epoxy resin (A) and cyanate resin (B).

5. An interlayer insulating resin film including an adhesive auxiliary layer, with an adhesive auxiliary layer provided on one surface of the interlayer insulating resin film, wherein the interlayer insulating resin film comprises epoxy resin (A), cyanate resin (B), and dicyandiamide (C), wherein the adhesive auxiliary layer comprises epoxy resin (a), cyanate resin (b), and inorganic filler (c).

6. The interlayer insulating resin film including an adhesive auxiliary layer according to claim 5, further comprising a support body provided on the opposite surface from the surface on which the interlayer insulating resin film of the adhesive auxiliary layer is provided.

7. A printed circuit board, comprising an interlayer insulating resin film comprising epoxy resin (A), cyanate resin (B), and dicyandiamide (C), or the interlayer insulating resin film having an adhesive auxiliary layer with an adhesive auxiliary layer provided on one surface of the interlayer insulating resin film, wherein the adhesive auxiliary layer comprises epoxy resin (a), cyanate resin (b), and inorganic filler (c).
